(19) United States
(12) Patent Application Publication
(10) Pub. No.: US 2009/0191177 A1
(43) Pub. Date: Jul. 30, 2009

(54) Enantiomerically Pure (-) 2-[1-(7-methyl-2-(morpholin-4-yl)-4-oxo-4H-pyrido[1,2-A]pyrimidin-9-yl)ethylamino]benzoic Acid, Its Use In Medical Therapy, And A Pharmaceutical Composition Comprising It - 026

(76) Inventors: Ola Fjellstrom, Molndal (SE); David Gustafsson, Molndal (SE); Jan A. Lindberg, Molndal (SE); Shaun Jackson, Victoria (AU)

Correspondence Address: Pepper Hamilton LLP, 400 Berwyn Park, 899 Cassatt Road, Berwyn, PA, 19312-1183, US

(21) Appl. No.: 12/359,323

(22) Filed: Jan. 25, 2009

Related U.S. Application Data
(60) Provisional application No. 61/023,498, filed on Jan. 25, 2008.

(51) Int. Cl. C07D 471/04; A61K 31/5355; A61K 31/727; A61K 38/45; A61P 7/00; A61P 9/00

(52) U.S. Cl. 424/94.5; 544/119; 514/232.2; 514/56

(57) ABSTRACT

The present invention relates to enantiomerically pure (−) 2-[1-(7-methyl-2-(morpholin-4-yl)-4-oxo-4H-pyrido[1,2-a]pyrimidin-9-yl)ethylamino]benzoic acid or pharmaceutically acceptable salts thereof, it being in a solid state, its use in medical therapy, pharmaceutical composition comprising it, its use in the preparation of a medicament for use in a method for preventing or treating diseases, and its use in method for preventing or treating disease. The present invention relates to a selective inhibitor of phosphoinositide (PI) 3-kinase β and use of the selective inhibitor in e.g. anti-thrombotic therapy.

### FIELD OF THE INVENTION

The present invention relates to enantiomerically pure (−) 2-[1-(7-methyl-2-(morpholin-4-yl)-4-oxo-4H-pyrido[1,2-a]pyrimidin-9-yl)ethylamino]benzoic acid or pharmaceutically acceptable salts thereof, the enantiomerically pure (−) 2-[1-(7-methyl-2-(morpholin-4-yl)-4-oxo-4H-pyrido[1,2-a]pyrimidin-9-yl)ethylamino]benzoic acid being in a solid state, a process for its preparation, its use in medical therapy, a pharmaceutical composition comprising it, its use in the preparation of a medicament for use in a method for preventing or treating diseases, and its use in method for preventing or treating disease. The present invention is, for example, concerned with a new antithrombotic therapy and the enantiomerically pure (−) 2-[1-(7-methyl-2-(morpholin-4-yl)-4-oxo-4H-pyrido[1,2-a]pyrimidin-9-yl)ethylamino]benzoic acid useful for the new therapy. More particularly, the present invention relates to a selective inhibitor of phosphoinositide (PI) 3-kinase β and use of the selective inhibitor (i.e. (−) 2-[1-(7-methyl-2-(morpholin-4-yl)-4-oxo-4H-pyrido[1,2-a]pyrimidin-9-yl)ethylamino]benzoic acid) in antithrombotic therapy.

### BACKGROUND OF THE INVENTION

Platelets are specialised adhesive cells that play a fundamental role in the haemostatic process. Under normal conditions, platelets neither adhere to, nor are activated by the vascular endothelium. However, damage to the endothelium or disruption of plaque exposes the flowing blood to a variety of thrombogenic elements. Circulating platelets bear receptors of these thrombogenic elements. Upon vascular injury, platelets, via glycoprotein GPIbα receptor, adhere to von Willebrand factor (vWF) bound to collagen at the site of ruptured plaques (platelet adhesion), become activated (platelet activation), and release a number of substances that are either premade or produced upon platelet activation including adenosine diphosphate (ADP), serotonin, and thromboxane A2 (TxA2) etc., all of which act as platelet agonists and thus potentiate the initial weak adhesion-induced platelet activation. In addition thrombin, which also is a potent platelet agonist, is generated by the coagulation cascade stimulated at a site of injury. One of the main functional responses to all these platelet agonists is transformation of the integrin α_{IIb}β₃ (GP IIb/IIIa) into its active conformation on the platelet surface. Once in its active conformation, these integrins will serve as receptors of fibrinogen bridges that link the platelets together (platelet aggregation) and subsequent thrombus formation.

Thus, sudden rupturing or fissuring of advanced atherosclerotic plaques causes an exaggerated platelet adhesion/aggregation response, which commonly leads to the formation of vaso-occlusive platelet thrombi. The formation of these thrombi in the coronary or cerebral circulation leads to acute myocardial infarction and stroke, respectively, which combined represent the leading causes of death in the industrialized world. Platelet thrombus formation also leads to a number of other clinical states including unstable angina, sudden death, transient ischemic attacks, amaurosis fugax, and acute ischemia of limbs and internal organs.

A number of factors that contribute to increase of thrombogenic potential of ruptured plaques include (1) the high reactivity of adhesive substrates in the plaque, (2) the presence of tissue factor in the lesion, and (3) the indirect platelet activating effects of high shear caused by narrowing of the vessel lumen by the atherothrombotic process.

The existing anti-thrombotic therapies mainly target one or more key steps in the thrombotic process. That is, anti-coagulants and anti-platelet agents are frequently used to alleviate thrombosis. Pathological thrombus formation can be minimized or eliminated in many instances by administering a suitable anti-coagulant, including one or more of a coumarin derivative (e.g., warfarin and dicumarol) or a charged polymer (e.g., heparin, hirudin or hirulog), or through the use of an anti-platelet agent (e.g, aspirin, clopidogrel, ticlopidine, dipyridimole, or one of several GPIIb/IIIa receptor antagonists). Anti-coagulants and platelet inhibitors suffer from a significant limitation, however, due to side effects such as hemorrhaging, re-occlusion, “white-clot” syndrome, irritation, birth defects, thrombocytopenia, and hepatic dysfunction. Moreover, long-term administration of anti-coagulants and platelet inhibitors can particularly increase risk of life-threatening illness or hemorrhage.

Thus, to avoid the aforementioned drawbacks of the existing anti-thrombotic therapy, there exists a need to develop a new anti-thrombotic therapy selectively targeting a process that is critical to pathological thrombus formation without interfering with normal haemostasis.

Rheological disturbances (high shear and turbulent flow) play a major role in promoting pathological thrombosis, and thus one such strategy would be to attenuate the platelet activating effects of high shear stress by targeting mechano-sensory elements in platelets. In WO2004016607 signaling events that are important for shear-induced platelet activation, but not for haemostasis, have been identified.

Moreover, the two major platelet adhesion receptors, GPIbα in the GPIb/V/IX glycoprotein complex and integrin α_{IIb}β₃, possess unique mechano-sensory functions relevant to platelet activation under conditions of theological disturbances (high shear and rapid accelerations in shear). In WO2004016607 it is described that signaling through both receptors is regulated by rapid accelerations in shear rate (↑Δγ), inducing platelet activation through PI 3-kinase-dependent signaling processes.

Further in WO2004016607, it is elucidated a critical signaling mechanism regulating platelet activation under high shear conditions and, PI 3-kinase β is identified as an element that induces platelet activation under pathological blood flow conditions. Before WO2004016607 existing anti-platelet therapies that block specific platelet adhesion receptors did not discriminate between pathological and normal haemostatic platelet activation. Therefore, the disclosure in WO2004016607, that selective inhibition of PI 3-kinase β could prevent platelet activation induced by pathological increases in shear rate, without affecting platelet activation induced by physiological agonists, provided a novel and specific approach to anti-thrombotic therapy, including new chemical compounds for such therapy. Further, it is also stressed, as shear-dependent platelet adhesion and activation is important in arterial thrombus formation, that PI 3-kinase β is an important target for therapeutic intervention in cardiovascular diseases generally.

Further WO2004016607 provides a method of disrupting platelet aggregation and adhesion occurring under high shear conditions, and a method for inhibiting platelet activation induced by shear, where both methods comprise the administering of a selective PI 3-kinase β inhibitor. WO2004016607 also provides an antithrombotic method comprising administering an effective amount of a selective PI 3-kinase β inhibitor. According to the method, specific inhibition of thrombosis can be obtained without affecting normal haemostasis by targeting PI 3-kinase β that is important for shear-induced platelet activation. Said antithrombotic method therefore does not involve side effects caused by disruption of normal haemostasis, such as extending of bleeding time.

Furthermore, in WO2004016607 a “selective PI 3-kinase β inhibitor” compound is understood to be more selective for PI 3-kinase β than compounds conventionally and generally designated PI 3-kinase inhibitors such as LY294002 or wortmannin. It is preferred in WO2004016607 that a selective PI 3-kinase β inhibitor is at least about >10-fold, more preferably >20-fold, more preferably >30-fold, selective for inhibition of PI 3-kinase β relative to other class I PI 3-kinase isoforms in a biochemical assay. Such other Type I PI3-kinases include PI 3-kinase α,γ and δ.

The compound 2-[1-(7-methyl-2-(morpholin-4-yl)-4-oxo-4H-pyrido[1,2-a]pyrimidin-9-yl)ethylamino]benzoic acid, which is a selective inhibitor of phosphoinositide (PI) 3-kinase β, is, together with other such inhibitors, described in WO2004016607. As further described in WO2004016607, the compound 2-[1-(7-methyl-2-(morpholin-4-yl)-4-oxo-4H-pyrido[1,2-a]pyrimidin-9-yl)ethylamino]benzoic acid may be useful in therapy, e.g. anti-thrombotic therapy. The compound 2-[1-(7-methyl-2-(morpholin-4-yl)-4-oxo-4H-pyrido[1,2-a]pyrimidin-9-yl)ethylamino]benzoic acid has an asymmetric center, i.e. the compound exists as two enantiomers. It is desireable to obtain compounds with improved activity, pharmacokinetic and/or metabolic properties. The present invention provides such a compound which is a single enantiomer of 2-[1-(7-methyl-2-(morpholin-4-yl)-4-oxo-4H-pyrido[1,2-a]pyrimidin-9-yl)ethylamino]benzoic acid.

### DESCRIPTION OF THE DRAWINGS

FIG. 1 shows the X-ray powder diffraction pattern of the (−)-enantiomer of 2-[1-(7-methyl-2-(morpholin-4-yl)-4-oxo-4H-pyrido[1,2-a]pyrimidin-9-yl)ethylamino]benzoic acid, i.e. (−) 2-[1-(7-methyl-2-(morpholin-4-yl)-4-oxo-4H-pyrido[1,2-a]pyrimidin-9-yl)ethylamino]benzoic acid.

### DESCRIPTION OF THE INVENTION

The present invention provides a new compound, i.e. enantiomerically pure

(−) 2-[1-(7-methyl-2-(morpholin-4-yl)-4-oxo-4H-pyrido[1,2-a]pyrimidin-9-yl)ethylamino]benzoic acid or pharmaceutically acceptable salts thereof.

The expression “enantiomerically pure” means (−) 2-[1-(7-methyl-2-(morpholin-4-yl)-4-oxo-4H-pyrido[1,2-a]pyrimidin-9-yl)ethylamino]benzoic acid essentially free from the other enantiomer, i.e. the (+)-enantiomer of 2-[1-(7-methyl-2-(morpholin-4-yl)-4-oxo-4H-pyrido[1,2-a]pyrimidin-9-yl)ethylamino]benzoic acid. Single enantiomers of 2-[1-(7-methyl-2-(morpholin-4-yl)-4-oxo-4H-pyrido[1,2-a]pyrimidin-9-yl)ethylamino]benzoic acid, including the (−) 2-[1-(7-methyl-2-(morpholin-4-yl)-4-oxo-4H-pyrido[1,2-a]pyrimidin-9-yl)ethylamino]benzoic acid of the present invention, have hitherto not been obtained. By means of the specific process, according to one aspect of the invention, of preparing the enantiomers of 2-[1-(7-methyl-2-(morpholin-4-yl)-4-oxo-4H-pyrido[1,2-a]pyrimidin-9-yl)ethylamino]benzoic acid, the pure enantiomers of the present invention are possible to obtain. The expression “enantiomerically pure” means e.g. ≧95% enantiomeric excess (ee) of one of the enantiomers of 2-[1-(7-methyl-2-(morpholin-4-yl)-4-oxo-4H-pyrido[1,2-a]pyrimidin-9-yl)ethylamino]benzoic acid.

The “enantiomerically pure” enantiomers are stable towards racemisation in pH 1-14.

Further, by means of said process, the pure enantiomers of 2-[1-(7-methyl-2-(morpholin-4-yl)-4-oxo-4H-pyrido[1,2-a]pyrimidin-9-yl)ethylamino]benzoic acid of the present invention may be obtained with high enantiomeric purity, e.g. ≧99.8% enantiomeric excess (ee), e.g. 99.9% ee of (−) 2-[(1R)-(7-Methyl-2-(morpholin-4-yl)-4-oxo-4H-pyrido[1,2-a]pyrimidin-9-yl)ethylamino]benzoic acid.

Furthermore, (−) 2-[(1R)-1-(7-methyl-2-(morpholin-4-yl)-4-oxo-4H-pyrido[1,2-a]pyrimidin-9-yl)ethylamino]benzoic acid and (+) 2-[(1s)-1-(7-Methyl-2-(morpholin-4-yl)-4-oxo-4H-pyrido[1,2-a]pyrimidin-9-yl)ethylamino]benzoic acid, respectively, or pharmaceutically acceptable salts thereof, may be provided with high enantiomeric purity.

The enantiomerically pure (−)-enantiomer of 2-[1-(7-methyl-2-(morpholin-4-yl)-4-oxo-4H-pyrido[1,2-a]pyrimidin-9-yl)ethylamino]benzoic acid has beneficial properties, for example, it is a selective PI 3-kinase β inhibitor as shown in Table 2.

Further, the enantiomerically pure (−) 2-[(1R)-1-(7-methyl-2-(morpholin-4-yl)-4-oxo-4H-pyrido[1,2-a]pyrimidin-9-yl)ethylamino]benzoic acid is in a neutral form. The neutral form may be more stable, easier to handle and store, easier to purify and easier to synthesise in a reproducible manner.

The invention further relates to enantiomerically pure

(−) 2-[1-(7-methyl-2-(morpholin-4-yl)-4-oxo-4H-pyrido[1,2-a]pyrimidin-9-yl)ethylamino]benzoic acid, or pharmaceutically acceptable salts thereof, being in a solid state which can be amorphous, at least partly crystalline or substantially crystalline. The crystalline form may be more stable, easier to handle and store, and easier to purify and easier to synthesise in a reproducible manner.

According to a further aspect of the invention, enantiomerically pure (−) 2-[(1R)-1-(7-methyl-2-(morpholin-4-yl)-4-oxo-4H-pyrido[1,2-a]pyrimidin-9-yl)ethylamino]benzoic acid, or pharmaceutically acceptable salts thereof, can exist in a solid state which can be at least partly crystalline or substantially crystalline. The crystalline form may be more stable, easier to handle and store, easier to purify and easier to synthesise in a reproducible manner.

In addition, by means of the specific process according to a further aspect of the invention, the pure enantiomer, i.e. the (−)-enantiomer of 2-[1-(7-methyl-2-(morpholin-4-yl)-4-oxo-4H-pyrido[1,2-a]pyrimidin-9-yl)ethylamino]benzoic acid, may be obtained in a solid state in a substantially crystalline form.

The (−)-enantiomer of 2-[1-(7-methyl-2-(morpholin-4-yl)-4-oxo-4H-pyrido[1,2-a]pyrimidin-9-yl)ethylamino]benzoic acid is characterised by having X-ray powder diffraction (XRPD) patterns having the d-values and relative intensities given in Table 1.

TABLE 1
(−) 2-[(1R)-1-(7-Methyl-2-(morpholin-4-yl)-4-oxo-4H-
pyrido[1,2-α]pyrimidin-9-yl)ethylamino]benzoic acid

| d value/Å | Intensity |
|---|---|
| 12.5 | w |
| 11.7 | w |
| 11.3 | w |
| 10.0 | w |
| 8.7 | w |
| 6.8 | vs |
| 6.2 | w |
| 6.1 | m |
| 5.9 | vs |
| 5.7 | w |
| 5.5 | m |
| 5.3 | w |
| 5.24 | m |
| 5.16 | w |
| 4.98 | m |
| 4.85 | m |
| 4.76 | w |
| 4.66 | w |
| 4.54 | w |
| 4.41 | m |
| 4.26 | vs |
| 4.06 | m |
| 3.91 | vs |
| 3.74 | m |
| 3.70 | m |
| 3.63 | w |
| 3.42 | m |
| 3.34 | w |
| 3.31 | w |
| 3.22 | m |
| 2.95 | w |
| 2.89 | w |
| 2.75 | vw |
| 2.71 | vw |
| 2.65 | w |
| 2.62 | w |
| 2.57 | w |
| 2.43 | w |
| 2.39 | w |
| 2.26 | w |
| 2.22 | w |
| 2.13 | w |
| 2.07 | w |

The X-ray powder diffraction (XRPD) patterns in these were obtained in Bragg-Bretano geometry. Absolute intensities were less accurate and therefore replaced with relative intensities:

vs=50-100, s=20-50, m=5-20, w=1-5, and vw<1.

The X-ray diffraction analysis was performed according to standard methods, which can be found in e.g. Kitaigorodsky, A. I. (1973), Molecular Crystals and Molecules, Academic Press, New York; Bunn, C. W. (1948), Chemical Crystallography, Clarendon Press, London; or Klug, H. P. & Alexander, L. E. (1974), X-Ray Diffraction Procedures, John Wiley & Sons, New York. X-ray powder diffraction pattern data were corrected by using corundum as an internal reference and measured with variable slits.

The invention relates to the pure enantiomer (−) 2-[(1R)-1-(7-methyl-2-(morpholin-4-yl)-4-oxo-4H-pyrido[1,2-a]pyrimidin-9-yl)ethylamino]benzoic acid having XRPD peaks at the following approximate d-values: 6.8, 5.9 and 3.91 Å.

Furthermore, the invention relates to the pure enantiomer (−) 2-[(1R)-1-(7-methyl-2-(morpholin-4-yl)-4-oxo-4H-pyrido[1,2-a]pyrimidin-9-yl)ethylamino]benzoic acid having XRPD peaks at the following approximate d-values: 6.8, 6.1, 5.9, 4.98, 4.41, 4.26 and 3.91 Å.

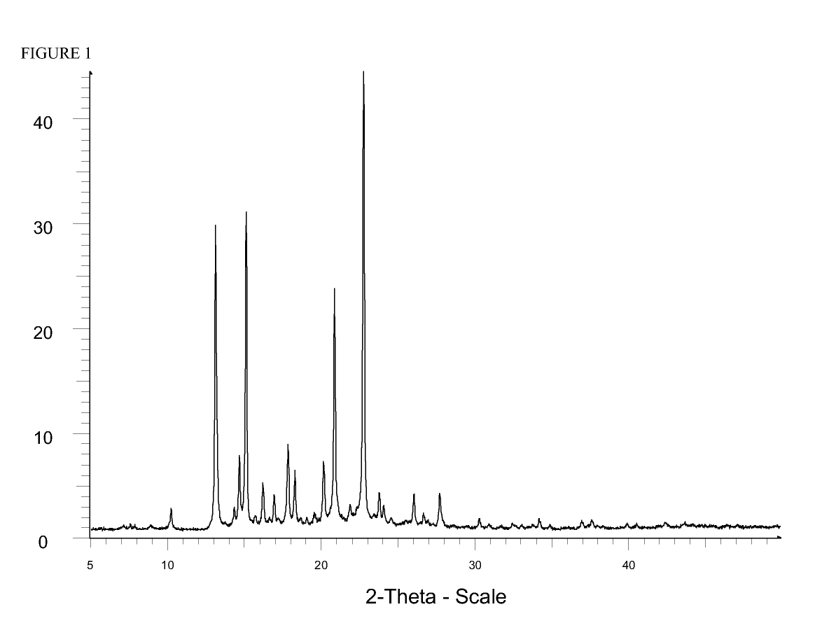

Further, the invention relates to (−) 2-[(1R)-1-(7-methyl-2-(morpholin-4-yl)-4-oxo-4H-pyrido[1,2-a]pyrimidin-9-yl)ethylamino]benzoic acid having an XRPD-diffractogram essentially as shown in FIG. 1.

In a further aspect, the invention relates to processes for the preparation of pure enantiomers of 2-[1-(7-methyl-2-(morpholin-4-yl)-4-oxo-4H-pyrido[1,2-a]pyrimidin-9-yl)ethylamino]benzoic acid which processes may include separation by fractional crystallisation or separation by chromatography.

The specific process for the preparation of pure enantiomers of 2-[1-(7-methyl-2-(morpholin-4-yl)-4-oxo-4H-pyrido[1,2-a]pyrimidin-9-yl)ethylamino]benzoic acid, which is described in the examples, comprises separation of the two enantiomers of methyl 2-{[1-(7-methyl-2-morpholin-4-yl-4-oxo-4H-pyrido[1,2-a]pyrimidin-9-yl)ethyl]amino}benzoate by chiral chromatography, followed by hydrolysis of the enantiomerically pure methyl esters and crystallisation.

It is also an object of the present invention to provide a method for preventing or treating cardiovascular disease, for example coronary artery occlusion, stroke, acute coronary syndrome, acute myocardial infarction, restenosis, atherosclerosis, and/or unstable angina, by administering an effective amount of a selective PI 3-kinase β inhibitor to a patient in need thereof. In this method, the use of the selective PI 3-kinase β inhibitor enables to avoid side effects caused by disruption of normal haemostasis, as measured by e.g. a prolongation of the cutaneous bleeding time.

The pure enantiomer, as described herein, or a pharmaceutically acceptable salt thereof, may be useful in therapy, especially adjunctive therapy, particularly it is indicated for use as: inhibitor of platelet activation adhesion/aggregation and degranulation, promoter of platelet disaggregation, anti-thrombotic agent or in the treatment or prophylaxis of thrombotic disorders. Examples of disorders associated with thrombosis or increased risk of thrombosis are unstable angina, myocardial infarction, thrombotic or embolic stroke, transient ischaemic attacks, peripheral vascular disease, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, pulmonary embolism, venoocclusive disease, haematological conditions such as myeloproliferative disease, including thrombocythaemia, sickle cell disease, percutaneous coronary interventions (PCI) or interventions in other vessels, stent placement, endarterectomy, coronary and other vascular graft surgery, thrombotic complications of surgical or mechanical damage such as tissue salvage following accidental or surgical trauma, reconstructive surgery including skin and muscle flaps, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in inflammatory conditions such as asthma and chronic obstructive pulmonary disease (COPD), in which platelets and platelet-derived factors are implicated in the immunological disease process. Conditions when blood is in contact with foreign surfaces in the body, e.g. in patients with biological or mechanical heart valves, indwelling permanent catheters, or when blood is in contact with foreign surfaces outside the body, e.g. in haemodialysis, plasmapheresis, cardio-pulmonary bypass and extracorporeal membrane oxygenation, or to facilitate thrombolysis or prevent re-occlusion after thrombolysis when thrombolysis is used in conditions like myocardial infarction, stroke, pulmonary embolism, deep venous thrombosis and catheter occlusions. Examples of increased platelet activation and aggregation that ex vivo, mechanically or by other means, are e.g. for the preservation of blood products, e.g. platelet concentrates.

According to the invention there is further provided the use of pure enantiomer, as described herein, or a pharmaceutically acceptable salt thereof, in the manufacture of a medicament for the treatment of the above disorders. In particular the pure enantiomer, as described herein, or a pharmaceutically acceptable salt thereof, may be useful for treating the above-described disorders. The invention also provides a method of treatment of the above disorders which comprises administering to a patient suffering from such a disorder a therapeutically effective amount of the pure enantiomer, as described herein, or a pharmaceutically acceptable salt thereof.

The invention further relates to use of the pure enantiomer, as described herein, or a pharmaceutically acceptable salt thereof, in the preparation of a medicament for use in a method for preventing or treating cardiovascular disease.

The invention also relates to the pure enantiomer, or a pharmaceutically acceptable salt thereof, for use in a method for preventing or treating cardiovascular disease, e.g. a method of antithrombosis.

Furthermore, the invention relates to a method of antithrombosis which involves administration of the pure enantiomer, as described herein, or a pharmaceutically acceptable salt thereof.

The invention also relates to a method for preventing or treating cardiovascular disease in a warm-blooded animal comprising administering an effective amount of the pure enantiomer, as described herein, or a pharmaceutically acceptable salt thereof.

The present invention also contemplates a method for inhibiting phosphoinositide 3-kinase β in a patient, comprising administering to a patient an amount of the pure enantiomer, as described herein, or a pharmaceutically acceptable salt thereof, effective in inhibiting the phosphoinositide 3-kinase β in the patient.

Furthermore, the invention relates to use of the pure enantiomer, as described herein, or a pharmaceutically acceptable salt thereof, in the preparation of a medicament for use in a method for preventing or treating respiratory disease.

The invention also relates to the pure enantiomer, or a pharmaceutically acceptable salt thereof, for the use in a method for preventing or treating respiratory disease.

The present invention also contemplates a method for preventing or treating respiratory disease in a warm-blooded animal comprising administering an effective amount of the pure enantiomer, as described herein.

Further, it is also known that PI 3-kinases contribute to tumourigenesis by one or more of the effects of mediating proliferation of cancer and other cells, mediating angiogenic events and mediating the motility, migration and invasiveness of cancer cells. The pure enantiomer of the present invention may possess potent anti-tumour activity which it is believed to obtain by way of inhibition of one or more of the Class I PI 3-kinases (such as the Class Ia PI 3-kinases and/or the Class Ib PI 3-kinase) and/or a PI3 kinase-related protein kinase (such as a DNA-PK, ATM or mTOR) that are involved in the repair of double stranded DNA-breaks (DNA-PK and ATM) and the signal transduction steps which lead to the proliferation and survival of tumour cells and the invasiveness and migratory ability of metastasising tumour cells (mTOR).

Accordingly, the pure enantiomer, as described herein, may be of value as anti-tumour agents, in particular as a selective inhibitor of the proliferation, survival, motility, dissemination and invasiveness of mammalian cancer cells leading to inhibition of tumour growth and survival and to inhibition of metastatic tumour growth. Particularly, the pure enantiomer of the present invention may be of value as an anti-proliferative and anti-invasive agent in the containment and/or treatment of solid tumour disease. Particularly, the pure enantiomer, as described herein, may be expected to be useful in the prevention or treatment of those tumours which are sensitive to inhibition of one or more of the multiple PI 3-kinases such as the Class Ia PI 3-kinases and the Class Ib PI 3-kinase that are involved in the signal transduction steps which lead to the proliferation and survival of tumour cells and the migratory ability and invasiveness of metastasising tumour cells. Further, the pure enantiomer, of the present invention, may be expected to be useful in the prevention or treatment of those tumours which are mediated alone or in part by inhibition of PI 3-kinases such as the Class Ia PI 3-kinases and the Class Ib PI 3-kinase, i.e. the pure enantiomer, as described herein, may be used to produce a PI 3-kinase inhibitory effect in a warm-blooded animal in need of such treatment.

Further, the pure enantiomer, as described herein, being an inhibitor of PI 3-kinase activity could be of therapeutic value for treatment of, for example, cancer of the breast, colorectum, lung (including small cell lung cancer, non-small cell lung cancer and bronchioalveolar cancer) and prostate, as well as of cancer of the bile duct, bone, bladder, head and neck, kidney, liver, gastrointestinal tissue, oesophagus, ovary, pancreas, skin, testes, thyroid, uterus, cervix and vulva, and of leukaemias (including acute lymphoctic leukaemia (ALL) and chronic myelogenous leukaemia (CML)), multiple myeloma and lymphomas.

According to the invention, there is further provided the use of pure enantiomer, as described herein, or a pharmaceutically acceptable salt thereof, in the manufacture of a medicament for the treatment of all disorders described herein.

The invention also provides a method of treatment of all disorders described herein which comprises administering to a patient suffering from such a disorder a therapeutically effective amount of the pure enantiomer, as described herein, or a pharmaceutically acceptable salt thereof.

Further, the invention relates to use of the pure enantiomer, as described herein, or a pharmaceutically acceptable salt thereof, in the preparation of a medicament for use in a method for preventing or treating cancer.

Further, the invention also relates to the pure enantiomer, or a pharmaceutically acceptable salt thereof, for use in a method for preventing or treating cancer.

The present invention also contemplates a method for preventing or treating cancer in a warm-blooded animal comprising administering an effective amount of the pure enantiomer, as described herein.

The invention also relates to use of the pure enantiomer, as described herein, or a pharmaceutically acceptable salt thereof, in the preparation of a medicament for use in a method for preventing or treating disease linked to disordered white blood cell function.

Further, the invention also relates to the pure enantiomer, or a pharmaceutically acceptable salt thereof, for use in a method for preventing or treating disease linked to disordered white blood cell function.

The present invention also contemplates a method for preventing or treating disease linked to disordered white blood cell function in a warm-blooded animal comprising administering an effective amount of the pure enantiomer, as described herein.

Thus, it is yet another object of the present invention to provide a method of inhibiting PI 3-kinase β comprising administering to the patient an amount of the pure enantiomer, as described herein, or a pharmaceutically acceptable salt thereof, wherein the amount is effective in inhibiting the PI 3-kinase β in the patient.

The pure enantiomer, as described herein, being an inhibitor of PI 3-kinase, also has potential therapeutic uses in a variety of other disease states. For example, PI 3-kinase plays an important role in promoting smooth muscle proliferation in the vascular tree, i.e. vascular smooth muscle cells, Thyberg, 1998, European Journal of Cell Biology 76(1):33-42, and in the lungs (airway smooth muscle cells), Krymskaya, V. P., BioDrugs, 2007. 21(2): 85-95. Excessive proliferation of vascular smooth muscle cells plays an important role in the formation of atherosclerotic plaques and in the development of neointimal hyperplasia following invasive vascular procedures, Scwartz et al., 1984, Progress in Cardiovascular Disease 26:355-372; Clowes et al., 1978, Laboratory Investigations 39:141-150. Moreover, excessive proliferation of airway smooth muscle cells leads to the development of COPD in the setting of asthma and chronic bronchitis. Inhibitors of PI 3-kinase activity therefore may be used to prevent vascular restenosis, atherosclerosis, and COPD.

PI 3-kinases also play an important role in regulating tumor cells and in the propensity of these cells to undergo apoptosis growth (Sellers et al., 1999, The Journal of Clinical Investigation 104:1655-1661). Additionally, uncontrolled regulation of the PI 3-kinase lipid products PI(3,4,5)P₃ and PI(3,4)P₂ by the lipid phosphatase PTEN plays an important role in progression of a number of malignant tumors in humans (Leevers et al., 1999, Current Opinion in Cell Biology 11:219-225). Therefore, the pure enantiomer, as described herein, being an inhibitor of PI 3-kinase, may be used to treat neoplasms in humans.

PI 3-kinase also plays an important role in leukocyte function (Fuller et al., 1999, The Journal of Immunology 162(11):6337-6340; Eder et al., 1998, The Journal of Biological Chemistry 273(43):28025-31) and lymphocyte function (Vicente-Manzanares et al., 1999, The Journal of Immunology 163(7):4001-4012). For example, leukocyte adhesion to inflamed endothelium involves activation of endogenous leukocyte integrins by a PI 3-kinase-dependent signaling process. Furthermore, oxidative burst (Nishioka et al., 1998, FEBS Letters 441(1):63-66 and Condliffe, A. M., et al., Blood, 2005. 106(4): 1432-40) and cytoskeletal reorganization (Kirsch et al., 1999, Proceedings National Academy of Sciences USA 96(11):6211-6216) in neutrophils appears to involve PI 3-kinase signaling. Neutrophil migration and directional movement are also dependent on PI3K activity (Camps, M., et al., Nat Med, 2005. 11(9): p. 936-43 and Sadhu, C., et al., J Immunol, 2003. 170(5): 2647-54). Thus, inhibitors of PI 3-kinase may be useful in reducing leukocyte adhesion and activation at sites of inflammation and therefore may be used to treat acute and/or chronic inflammatory disorders. PI 3-kinase also plays an important role in lymphocyte proliferation and activation, Fruman et al., 1999, Science 283 (5400): 393-397. Given the important role of lymphocytes in auto-immune diseases, an inhibitor of PI 3-kinase activity may be used in the treatment of such disorders.

The efficacy of a compound, e.g. the pure enantiomer, as described herein, as an inhibitor of an enzyme activity can be established, for example, by determining the concentrations at which the compound inhibits the activity to a predefined extent and then comparing the results. Typically, the preferred determination is the concentration that inhibits 50% of the activity in a biochemical assay, i.e. the 50% inhibitory concentration or “IC₅₀”. IC₅₀ can be determined using conventional techniques known in the art.

Further, the present invention also relates to a combination comprising the pure enantiomer, as described herein, or a pharmaceutically acceptable salt thereof, and any antithrombotic agent(s) with a different mechanism of action, wherein said antithrombotic agent(s) may be, for example, one or more of the following: the anticoagulants unfractionated heparin, low molecular weight heparin, other heparin derivatives, synthetic heparin derivatives (e.g. fondaparinux), vitamin K antagonists (e.g. warfarin), synthetic or biotechnological inhibitors of coagulation factors (e.g. synthetic thrombin, FVIIa, FXa, FXIa and FIXa inhibitors, and rNAPc2), the antiplatelet agents acetylsalicylic acid, dipyridamole, cilostazol, ticlopidine, clopidogrel, prasugrel, AZD6140, other inhibitors of ADP/ATP receptors (P2X1, P2Y1, P2Y12 ); thromboxane receptor and/or synthetase inhibitors; tirofiban, eptifibatide, abciximab or other GPIIb/IIIa antagonists, prostacyclin mimetics, phosphodiesterase inhibitors, inhibitors of protease activated receptors (PAR1 or PAR4) like the PAR1 antagonist SCH 530348, p-selectin antagonists, GPVI antagonists, GPIbα-vWF-collagen interaction inhibitors, EP3 receptor antagonists and fibrinolysis stimulating agents that work by inhibiting carboxypeptidase U (CPU or TAFIa) or plasminogen activator inhibitor-1 (PAI-1).

Furthermore, the present invention relates to a combination comprising the pure enantiomer, as described herein, or a pharmaceutically acceptable salt thereof, and thrombolytics, e.g. one or more of tissue plasminogen activator (natural, recombinant or modified), streptokinase, urokinase, prourokinase, anisoylated plasminogen-streptokinase activator complex (APSAC), animal salivary gland plasminogen activators, microplasmin or other plasmin variants.

In the present methods for preventing or treating a disease condition, the effective amount of the pure enantiomer, as described herein, or a pharmaceutically acceptable salt thereof, may be administered in the form of a dose. In further embodiments, the dose may be in the form of a tablet (e.g. a tablet formulated for oral, sublingual, and buccal administration), a capsule (e.g. a capsule containing powder, liquid, or a controlled-release formulation), an intravenous formulation, an intranasal formulation, a formulation for muscular injection, a syrup, a suppository, an aerosol, a buccal formulation, a transdermal formulation, or a pessary. Further, the dose contains from about 5 to about 500 mg of the pure enantiomer, as described herein, or a pharmaceutically acceptable salt thereof, and even further contains from about 25 to about 300 mg of the pure enantiomer, as described herein, or a pharmaceutically acceptable salt thereof.

Another aspect of the present invention relates to a pharmaceutical composition containing the pure enantiomer, as described herein, or a pharmaceutically acceptable salt thereof, together with one or more pharmaceutically acceptable carriers and/or diluents. Below, the term “active ingredient” may be the pure enantiomer, as described herein, or a physiologically acceptable salt, solvate, or functional derivative thereof.

Administration of this pharmaceutical composition may be performed by any convenient means. Doses may be administered daily, weekly, monthly, or at other suitable time intervals, for example, by the oral, intravenous, intraperitoneal, intramuscular, subcutaneous, intradermal or suppository routes, or by implanting (e.g. using a slow-release formulation). If the pure enantiomer, as described herein, or a pharmaceutically acceptable salt thereof, may be administered in tablet form, the tablet may contain a binder e.g. tragacanth, corn starch, or gelatin; a disintegrating agent, e.g. alginic acid; and a lubricant, e.g. magnesium stearate.

The pharmaceutical compositions suitable for injectable use include sterile aqueous solutions or dispersions, and sterile powders for the extemporaneous preparation of sterile injectable solutions or dispersions, or may be in the form of a cream or other form suitable for topical application. The carrier can be a solvent or dispersion medium containing, for example, water, ethanol, polyol (e.g., glycerol, propylene glycol, and liquid polyethylene glycol, and the like), suitable mixtures thereof, and vegetable oils. The proper fluidity may be maintained, for example, by the use of a coating e.g. lecithin, by the maintenance of the required particle size in the case of dispersion, and by the use of superfactants. Prevention of contamination by microorganisms can be brought about by various antibacterial and antifungal agents, for example, parabens, chlorobutanol, phenol, sorbic acid, thimerosal or the like. It may be possible to include isotonic agents, for example sugars or sodium chloride. Prolonged absorption of the injectable compositions can be brought about by the use in the compositions of agents delaying absorption, for example, aluminum monostearate and gelatin.

Sterile injectable solutions may be prepared by incorporating the pure enantiomer, as described herein, or a pharmaceutically acceptable salt thereof, in the required amount in the appropriate solvent with various other ingredients as exemplified above, followed by filter sterilization. Generally, dispersions may be prepared by incorporating the sterilized active pure enantiomer into a sterile vehicle containing the basic dispersion medium and one or more of the above-described ingredients. In the case of sterile powders for the preparation of sterile injectable solutions, methods of preparation may be vacuum drying and freeze drying which may yield a powder of the pure enantiomer, as described herein, or a pharmaceutically acceptable salt thereof, plus any additional desired ingredients from previously sterile-filtered solutions thereof.

The pharmaceutical compositions may be orally administered, for example, with an inert diluent or with an assimilable edible carrier, may be enclosed in hard or soft shell gelatin capsule, may be compressed into tablets or may be incorporated directly with food. For oral administration, the pure enantiomer, as described herein, or a pharmaceutically acceptable salt thereof, may be incorporated with excipients, and may be used in the form of ingestible tablets, buccal tablets, troches, capsules, elixirs, suspensions, syrups, wafers, and the like. Such compositions and preparations may contain at least 1% by weight of the pure enantiomer, as described herein, or a pharmaceutically acceptable salt thereof. The percentage of the compositions and preparations may be varied and may be between about 5 to about 80% of the weight of the unit. The amount of the pure enantiomer, as described herein, or a pharmaceutically acceptable salt thereof, in such therapeutically useful compositions may be such that a suitable dosage will be obtained.

The tablets, troches, pills, capsules and the like may also contain a binder e.g. gum, acacia, corn starch, or gelatin; excipients e.g. dicalcium phosphate; a disintegrating agent e.g. corn starch, potato starch, alginic acid and the like; a lubricant e.g. magnesium stearate; and a sweetening agent e.g. sucrose, lactose or saccharin may be added or a flavoring agent e.g. peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup or elixir may contain the pure enantiomer, as described herein, or a pharmaceutically acceptable salt thereof, e.g. sucrose as a sweetening agent, e.g. methyl or propyl-parabens as preservatives, e.g. a dye and e.g. flavoring, for example, cherry or orange flavor. Of course, any material used in preparing any dosage unit form should be pharmaceutically pure and substantially non-toxic in the amounts employed. In addition, the active pure enantiomer may be incorporated into sustained-release preparations and formulations.

The invention is further described by reference, but not limited, to the following examples.

Experimental Section

Abbreviations:

DIPEA N,N-diisopropylethylamine

DPPP 1,3-bis(diphenylphosphino)propane

DMF N,N-dimethylformamide

HPLC high performance liquid chromatography

THF tetrahydrofurane

Ms methanesulfonyl

MTBE methyl tert-butylether

s singlet

d doublet

dd double doublet

dt doublet of triplets

t triplet

m multiplet

General Experimental Procedures

¹H NMR and ¹³C NMR spectra were obtained at 298 K on a Varian Unity Plus 400 mHz, or a Varian Inova 500 MHz. Chemical shifts are given in ppm with the solvent residual peak as internal standard: CDCl₃ δ7.26; DMSO-d₆ δ_{H} 2.50; δ_{C} 39.5 ppm. Optical rotations were recorded at 20° C. on a Perkin Elmer Model 341 polarimeter. Melting points were recorded with a Stuart Scientific SMP3 melting point apparatus. The ee of the title compounds was determined by analytical chiral chromatography on a 4.6×250 mm Chiralpak AS column eluted with MeOH/formic acid 100/0.1.

X-ray powder diffraction pattern data was measured on a Bruker D8Advance X-ray powder diffractometer, without internal references and with variable slits.

Chemical names (IUPAC) were generated using the software ACD/Name version 9.04.

9-Bromo-2-hydroxy-7-methyl-4H-pyrido[1,2-a]pyrimidin-4-one hydrochloride was prepared as described in WO2004016607.

### EXAMPLES

### Example 1

### (−) 2-[1-(7-Methyl-2-(morpholin-4-yl)-4-oxo-4H-pyrido[1,2-a]pyrimidin-9-yl)ethylamino]benzoic acid, probably (−) 2-{[(1R)-1-(7-methyl-2-morpholin-4-yl-4-oxo-4H-pyrido[1,2-a]pyrimidin-9-yl)ethyl]amino}benzoic acid

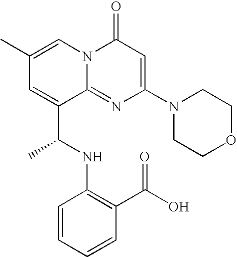

### a) 9-Bromo-7-methyl-2-morpholin-4-yl-4H-pyrido[1,2-a]pyrimidin-4-one

To a stirred slurry of 9-bromo-2-hydroxy-7-methyl-4H-pyrido[1,2-a]pyrimidin-4-one hydrochloride (407 g, 1.40 mol) in dry THF (4 L) at 5° C. under a N₂-atm, triethylamine (259 g, 2.56 mol) was added over 10 min. MsCl (259 g, 2.26 mol) was added over 30 min and the resulting mixture stirred for 2.5 h at 5° C. Morpholine (388 g, 4.45 mol) was added and the resulting mixture stirred at 60° C. for 6 h. Water (8.2 L) was added and the resulting mixture stirred at 65° C. for 3 h. The mixture was cooled to 20° C. during 4 h and stirred at 20° C. over night. The product was filtered off, washed with water (2.0 L+1.5 L) and dried under vacuum at 0-1 mbar and 40° C. to yield 426 g (94%) of the subtitle compound.

¹H NMR (400 MHz, CDC₃) δ 8.71 (s, 1H), 7.84 (s, 1H), 5.60 (s, 1H), 3.89-3.60 (m, 8H), 2.34 (s, 3H).

### b) 9-Acetyl-7-methyl-2-morpholin-4-yl-4H-pyrido[1,2-a]pyrimidin-4-one

A stirred slurry of 9-bromo-7-methyl-2-morpholin-4-yl-4H-pyrido[1,2-a]pyrimidin-4-one (677 g, 2.09 mol), K₂CO₃ (375 g, 2.71 mol) in DMF (3.0 L) and water (400 mL) at 40° C. under a N₂-atm in a 10 L reactor was degassed by evacuating and filling the reactor with nitrogen. n-Butyl vinyl ether (1267 g, 12.6 mol) was added and the resulting mixture was degassed one more time. A suspension of DPPP (69.0 g, 0.17 mol) and Pd(OAc)₂ (9.24 g, 0.041 mol) in DMF (340 mL) was added and the resulting mixture stirred at 90° C. for 2 days. The reaction mixture was concentrated under vacuum at 90° C. to a total volume of 2.5 L. H₂O (9 L) was added and the resulting suspension was stirred for 2 h at 20° C. Solids were filtered off and transferred to a 25 L reactor. Water (15 L) and 3.6 M HCl (5 L) were added and the mixture was slowly heated to 37° C. during 1 h. The almost clear solution was filtered and the filtrate was returned to the 25 L reactor. The product was precipitated by addition of 45% NaOH (950 mL) until pH 6, and the resulting slurry stirred at 20° C. over night. The product was filtered off, washed with water (2×4 L) and dried under vacuum at 40° C. and 0-1 mbar to yield 531 g (89%) of the subtitle compound.

¹H NMR (400 MHz, CDCl₃) δ 8.86 (s, 1H), 7.84 (s, 1H), 5.66 (s, 1H), 3.83-3.75 (m, 4H), 3.66-3.59 (m, 4H), 2.77 (s, 3H), 2.36 (s, 3H).

### c) 9-(1-Hydroxyethyl)-7-methyl-2-morpholin-4-yl-4H-pyrido[1,2-a]pyrimidin-4-one

To a stirred slurry of 9-acetyl-7-methyl-2-morpholin-4-yl-4H-pyrido[1,2-a]pyrimidin-4-one (502 g, 1.75 mol) in MeOH (4.7 L), NaBH₄ (64.9 g, 1.72 mol) was added over 1 h. The resulting mixture was stirred for 1 h, H₂O (1 L) was added and MeOH distilled off under vacuum at 70° C. to a total volume of 2 L. Water (3 L) was added and the resulting slurry was stirred at 50° C. for 30 min, cooled to 2° C. during 6 h and stirred at 2° C. over night. The product was filtered off, washed with cold water (2×1 L) and dried in a vacuum oven at 40° C. and 0-1 mbar to give 404 g (80%) of the subtitle compound.

¹H NMR (400 MHz, CDCl₃) δ 8.63 (s, 1H), 7.50 (s, 1H), 5.64 (s, 1H), 5.23-5.16 (m, 1H), 3.82-3.75 (m, 4H), 3.63-3.55 (m, 4H), 2.33 (s, 3H), 1.60 (d, 3H).

### d) 2-{[1-(7-Methyl-2-morpholin-4-yl-4-oxo-4H-pyrido[1,2-a]pyrimidin-9-yl)ethyl]amino}benzoic acid

To a stirred solution of 9-(1-hydroxyethyl)-7-methyl-2-morpholin-4-yl-4H-pyrido[1,2-a]pyrimidin-4-one (338 g, 1.17 mol) in CH₂Cl₂ (4.0 L), PBr₃ (1M in CH₂Cl₂, 600 mL) was added. The resulting mixture was stirred at 40° C. for 2.5 h. 2-Aminobenzoic acid (192 g, 1.40 mol) end triethylamine (0.66 L, 4.74 mol) were sequentially added and the resulting mixture stirred at 40° C. over night. Water (2.0 L) was added, the mixture was stirred for 5 min and the phases were separated. The organic phase was concentrated to a volume of ˜1.0 L. To the stirred residue at 20° C. acetone (3.5 L) and 4M HCl (800 mL) were added and the resulting slurry was stirred over night. The product was filtered off, washed with acetone (1.0 L) and water (2.0 L) and dried in a vacuum oven at 40° C., 0-1 mbar, to give 330 g (69%) of the subtitle compound.

¹H NMR (400 MHz, DMSO-d₆) δ 8.54 (s, 1H), 8.40 (d, 1H), 7.80 (dd, 1H), 7.60 (d, 1H), 7.23 (dt, 1H), 6.54 (t, 1H), 6.37 (d, 1H), 5.65 (s, 1H), 5.22 (m, 1H), 3.69 (m, 4H), 3.63 (m, 4H), 2.24 (s, 3H), 1.58 (d, 3H).

### e) (−) Methyl 2-{[(1R)-1-(7-methyl-2-morpholin-4-yl-4-oxo-4H-pyrido[1,2-a]pyrimidin-9-yl)ethyl]amino}benzoate

To a stirred slurry of 2-{[1-(7-methyl-2-morpholin-4-yl-4-oxo-4H-pyrido[1,2-a]pyrimidin-9-yl)ethyl]amino}benzoic acid (115.4 g, 0.28 mol) in DMF (1.0 L) were added DIPEA (80 mL, 0.46 mol) and MeI (25 mL, 0.40 mol). The resulting mixture was stirred over night at rt. MTBE (1 L) and H₂O (2 L) were added. The mixture was stirred for 30 min and the phases were separated. The aqueous layer was extracted twice with MTBE (0.6+0.5 L) and the combined organic phases were washed with 0.05M NaHCO₃ (2×0.5 L) and 0.4 L H₂O. The organic phase was concentrated and the enantiomers separated by chiral chromatography on a Chiralpak AS HPLC-column eluted with heptane/EtOH 20:80. The slower eluting compound was collected to yield 48 g (99.4% ee) of the subtitle compound.

¹H NMR (400 MHz, CDC₃) δ 8.64 (s, 1H), 8.23 (d, 1H), 7.92 (dd, 1H), 7.50 (d, 1H), 7.19 (dt, 1H), 6.58 (t, 1H), 6.27 (d, 1H), 5.66 (s, 1H), 5.30-5.22 (m, 1H), 3.91 (s, 3H), 3.83-3.78 (m, 4H), 3.69-3.63 (m, 4H), 2.24 (s, 3H), 1.63 (d, 3H).

### f) (−) 2-{[(1R)-1-(7-Methyl-2-morpholin-4-yl-4-oxo-4H-pyrido[1,2-a]pyrimidin-9-yl)ethyl]amino}benzoic acid

To a stirred solution of (−) methyl 2-{[(1R)-1-(7-methyl-2-morpholin-4-yl-4-oxo-4H-pyrido[1,2-a]pyrimidin-9-yl)ethyl]amino}benzoate (5.22 g, 12.4 mmol) in THF (35 mL) and MeOH (35 mL), NaOH (2.7 g, 67.5 mmol) dissolved in H₂O (35 mL) was added. After 3 days at rt, the mixture was concentrated under vacuum until ˜35 mL remained, diluted with H₂O (200 mL) and washed with CH₂Cl₂ (50 mL). The aqueous layer was acidified with 1M HCl (70 mL) and extracted with CH₂Cl₂ (50 mL). The organic layer was washed with brine, dried with MgSO₄, filtered and concentrated to ˜5 mL. Acetone (15 mL) was added to the residue and the mixture stirred over night. The crystalline material was filtered off and washed with acetone. Traces of acetone and CH₂Cl₂ were removed by an additional recrystallisation from EtOH/H₂O to give 2.94 g (58%) of the title compound.

¹H NMR (500 MHz, DMSO-d₆) δ 8.53 (s, 1H), 8.39 (d, 1H), 7.79 (dd, 1H), 7.59 (d, 1H), 7.21 (dt, 1H), 6.52 (t, 1H), 6.36 (d, 1H), 5.65 (s, 1H), 5.21 (m, 1H), 3.68 (m, 4H), 3.62 (m, 4H), 2.22 (s, 3H), 1.57 (d, 3H);

³C NMR (500 MHz, DMSO-d₆) δ 170.0, 159.7, 157.5, 149.4, 146.9, 136.6, 135.5, 134.5, 131.7, 123.3, 122.1, 114.7, 111.9, 110.4, 80.1, 65.8 (2C), 47.5, 44.2 (2C), 21.4, 17.6;

LC-MS [M+H]⁺409.19

(99.9% ee)

[α]_{D}=−449, (c 0.2, CH₃CN)

mp 245.2-245.6° C.

### Example 2

### (+) 2-[1-(7-Methyl-2-(morpholin-4-yl)-4-oxo-4H-pyrido[1,2-a]pyrimidin-9-yl)ethylamino]benzoic acid, probably (+) 2-{[(1S)-1-(7-methyl-2-morpholin-4-yl-4-oxo-4H-pyrido[1,2-a]pyrimidin-9-yl)ethyl]amino}benzoic acid

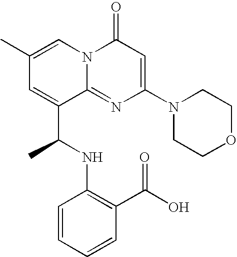

### a) (+) Methyl 2-{[(1S)-1-(7-methyl-2-morpholin-4-yl-4-oxo-4H-pyrido[1,2-a]pyrimidin-9-yl)ethyl]amino}benzoate

The faster eluting compound from step e in Example 1 was collected to give 51 g (99.8% ee) of the subtitle compound.

### b) (+) 2-{[(1S)-1-(7-Methyl-2-morpholin-4-yl-4-oxo-4H-pyrido[1,2-a]pyrimidin-9-yl)ethyl]amino}benzoic acid

The title compound was prepared from (+) methyl 2-{[(1S)-1-(7-methyl-2-morpholin-4-yl-4-oxo-4H-pyrido[1,2-a]pyrimidin-9-yl)ethyl]amino}benzoate (11,9 g, 28 mmol) by the same method as for (−) 2-{[(1R)-1-(7-methyl-2-morpholin-4-yl-4-oxo-4H-pyrido[1,2-a]pyrimidin-9-yl)ethyl]amino}benzoic acid in example 1, see 1f), to give 10.9 g (94%) of the title compound.

¹H NMR (400 MHz, DMSO-d₆) δ 8.53 (s, 1H), 8.39 (d, 1H), 7.79 (dd, 1H), 7.59 (d, 1H), 7.21 (dt, 1H), 6.52 (t, 1H), 6.36 (d, 1H), 5.65 (s, 1H), 5.21 (m, 1H), 3.68 (m, 4H), 3.62 (m, 4H), 2.22 (s, 3H), 1.57 (d, 3H);

¹³C NMR (DMSO-d₆) δ 170.0, 159.7, 157.5, 149.4, 146.9, 136.6, 135.5, 134.5, 131.7, 123.3, 122.1, 114.7, 111.9, 110.4, 80.1, 65.8 (2C), 47.5, 44.2 (2C), 21.4, 17.6;

(99.8% ee)

[α]_{D}=+443, (c 0.2, CH₃CN)

mp 244.0-244.5° C.

Assay of Enzyme Inhibition

The inhibition of PI3Kβ, PI3Kα, PI3Kγ and PI3Kδ was evaluated in an AlphaScreen based enzyme activity assay using human recombinant enzymes. The assay measures PI3K-mediated conversion of phophatidylinositol (4,5)bisphosphate (PIP2) to phosphatidylinositol (3,4,5)trisphosphate (PIP3). Biotinylated PIP3, a GST-tagged pleckstrin homology (PH) domain and the two AlphaScreen beads form a complex that elicits a signal upon laser excitation at 680 nm. The PIP3 formed in the enzyme reaction competes with the biotinylated PIP3 for binding to the PH domain thus reducing the signal with increasing enzyme product.

Ten different Compound concentrations were tested, and inhibition of PI3Kβ, PI3Kα, PI3Kγ and PI3Kδ, expressed as a per cent of maximal activity, was plotted versus inhibitor concentration.

Method

The Compound was dissolved in DMSO and added to 384 well plates. PI3Kβ, PI3Kα, PI3Kγ or PI3Kδ was added in a Tris buffer (50 mM Tris pH 7.6, 0.05% CHAPS, 5 mM DTT and 24 mM MgCl₂) and allowed to preincubate with the Compound for 20 minutes prior to the addition of substrate solution containing PIP2 and ATP. The enzyme reaction was stopped after 20 minutes by addition of stop solution containing EDTA and biotin-PIP3, followed by addition of detection solution containing GST-grp1 PH and AlphaScreen beads. Plates were left for a minimum of 5 hours in the dark prior to analysis. The final concentration of DMSO, ATP and PIP2 in the assay were, 0.8%, 4 μM and 40 μM, respectively.

Data Analysis

IC₅₀ values were calculated according to the equation, y=(a+((b−a)/(1+(x/IC₅₀)^{s}))), where y=% inhibition; a=0%; b=100%; s=the slope of the concentration−response curve; x=inhibitor concentration. Data are presented in Table 2.

Assay of Washed Platelet Aggregation (WPA)

Blood was collected from healthy volunteers by venipuncture using a Venflon needle 1.5*45 mm (17 GA, 1.77 IN). The first 2 ml of blood was discarded prior to collecting aliquotes into tubes containing acid citrate dextrose (ACD). One volume of ACD is required for six volumes of blood.

The anticoagulated blood was centrifuged for 15 min at 240×g to obtain platelet rich plasma (PRP). PRP was transferred to a new tube and centrifuged for 15 min at 2200×g. The supernatant was discarded and the platelet pellet re-suspended to 200000×10⁹/L in Tyrodes buffer (TB) containing 1 μM hirudin and 0.02 U/mL apyrase. The platelet suspension was left to rest at room temperature for 30 min. Just prior to time for assay, CaCl₂ was added to a final concentration of 2 mM. The Compound, or wortmannin, was dissolved in DMSO and added to a 96 well plate prior to the addition of the washed platelet suspension. The platelet suspension was preincubated with inhibitor for 5 min. Light absorption at 650 nm was recorded before and after a 5 min plate shake and referred to as recording 0 (R0) and R1. A mouse anti-human CD9 antibody was added (at a donor specific concentration) to each well prior to next 10 min plate shake and light absorption recording; R2.

A concentration response for CD9 antibody-induced aggregation in the absence or presence of 1 μM wortmannin (complete PI3K inhibition) was performed in each washed platelet suspension prior to testing of the Compound. The CD9 antibody concentration with the largest dependence of PI3K inhibition was chosen for the test.

Data Analysis

Light absorbance in wells with TB were subtracted from all readings before percent aggregation was calculated according the formula: [(R1−R2)/R1]×100=% aggregation. Spontaneous aggregation or pro-aggregatory effect of the inhibitor was evaluated by the same formula, [(R0−R1)/R0]×100=% aggregation. IC₅₀ values were calculated according to the equation, y=(a+((b−a)/(1+(x/IC₅₀)^{s}))), where y=Washed platelet aggregation; a=minimum aggregation; b=maximum aggregation; s=the slope of the concentration−response curve; x=inhibitor concentration. Data are presented in Table 2.

TABLE 2
IC₅₀ for PI3Kβ, PI3Kα, PI3Kγ and PI3Kδ and in WPA for each of the
enantiomers, as well as for the racemate
Table 2

| | | | | | Washed |
|---|---|---|---|---|---|
| | | | | | Platelet |
| | PI₃K_{β} | PI₃K_{α} | PI₃K_{γ} | PI₃K_{δ} | Aggregation |
| Compound Name | μM | | | | nM |
| Ex. 1 (−)2-[(1R)-1-(7-Methyl-2-(morpholin-4-yl)-4- | 0.021 | 1.4 | 1.2 | 0.08 | 6 |
| oxo-4H-pyrido[1,2-α]pyrimidin-9- | | | | | |
| yl)ethylamino]benzoic acid | | | | | |
| Ex. 2 (+)2-[(1S)-1-(7-Methyl-2-(morpholin-4-yl)-4- | 4.4 | 23 | 23 | 8 | 1653 |
| oxo-4H-pyrido[1,2-α]pyrimidin-9- | | | | | |
| yl)ethylamino]benzoic acid | | | | | |
| WO2004016607 | 0.110 | 6.8 | 3.7 | 0.8 | 10 |
| 2-[1-(7-Methyl-2-(morpholin-4-yl)-4-oxo-4H- | | | | | |
| pyrido[1,2-α]pyrimidin-9-yl)ethylamino]benzoic acid | | | | | |

1. Enantiomerically pure (−) 2-[1-(7-methyl-2-(morpholin-4-yl)-4-oxo-4H-pyrido[1,2-a]pyrimidin-9-yl)ethylamino]benzoic acid or a pharmaceutically acceptable salt thereof.

2. The pure enantiomer according to claim 1 characterized in that the enantiomer is in an enantiomeric excess (ee) of ≧95%.

3. The pure enantiomer according to claim 1 characterized in that the enantiomer is in an enantiomeric excess (ee) of ≧99.8%.

4. The pure enantiomer according to claim 1 characterized in that the enantiomer is in a solid state.

5. The pure enantiomer according claim 1 characterized in that the enantiomer is in a partly crystalline state.

6. The pure enantiomer according to claim 1 characterized in that the enantiomer is in a substantially crystalline state.

7. The pure enantiomer according to claim 1, characterized by having XRPD peaks at the following approximate d-values: 6.8, 5.9 and 3.91 Å.

8. The pure enantiomer according to any of claim 1, characterized by having XRPD peaks at the following approximate d-values: 6.8, 6.1, 5.9, 4.98, 4.41, 4.26 and 3.91 Å.

9. (−) 2-[(1R)-1-(7-Methyl-2-(morpholin-4-yl)-4-oxo-4H-pyrido[1,2-a]pyrimidin-9-yl)ethylamino]benzoic acid according to claim 7 characterized by having an XRPD-diffractogram essentially as shown in FIG. 1.

10. A process for preparing the pure enantiomer according to claim 1 which comprises separation of the two enantiomers of methyl 2-{[-1-(7-methyl-2-morpholin-4-yl-4-oxo-4H-pyrido[1,2-a]pyrimidin-9-yl)ethyl]amino}benzoate by chiral chromatography, followed by hydrolysis of the methyl ester and crystallisation.

11. (canceled)

12. A pharmaceutical composition comprising the pure enantiomer according to claim 1, or a pharmaceutically acceptable salt thereof, with a pharmaceutically acceptable diluent or carrier.

13-17. (canceled)

18. A method for preventing or treating cardiovascular disease in a warm-blooded animal comprising administering an effective amount of the pure enantiomer according to claim 1, or a pharmaceutically acceptable salt thereof.

19. A method for preventing or treating respiratory disease in a warm-blooded animal comprising administering an effective amount of the pure enantiomer according to claim 1, or a pharmaceutically acceptable salt thereof.

20. A method for preventing or treating cancer in a warm-blooded animal comprising administering an effective amount of the pure enantiomer according to claim 1, or a pharmaceutically acceptable salt thereof.

21. A method for preventing or treating disease linked to disordered white blood cell function in a warm-blooded animal comprising administering an effective amount of the pure enantiomer according to claim 1, or a pharmaceutically acceptable salt thereof.

22. A combination comprising the pure enantiomer according to claim 1, or a pharmaceutically acceptable salt thereof, and an antithrombotic agent with a different mechanism of action, wherein said antithrombotic agent is an anticoagulant or an antiplatelet agent, of.

23. A combination comprising the pure enantiomer according to claim 1, or a pharmaceutically acceptable salt thereof, and a thrombolytic.

24. 2-{[(1R)-1-(7-Methyl-2-morpholin-4-yl-4-oxo-4H-pyrido[1,2-a]pyrimidin-9-yl)ethyl]amino}benzoic acid, or a pharmaceutically acceptable salt thereof.

25. The combination according to claim 22 wherein the anticoagulant is chosen from unfractionated heparin, low molecular weight heparin, a heparin derivative, a synthetic heparin derivative, a vitamin K antagonist, and a synthetic or biotechnological inhibitor of a coagulation factor; and the antiplatelet agent is chosen from acetylsalicylic acid, dipyridamole, cilostazol, ticlopidine, clopidogrel, prasugrel, AZD6140, an inhibitor of an ADP/ATP receptor, a thromboxane receptor and/or synthetase inhibitor, tirofiban, eptifibatide, abciximab, another GPIIb/IIIa antagonist; a prostacyclin mimetic, a phosphodiesterase inhibitor, an inhibitor of a protease activated receptor, a p-selectin antagonist, a GPVI antagonist, a GPIbα-vWF-collagen interaction inhibitor, an EP3 receptor antagonist, and a fibrinolysis stimulating agent that inhibits carboxypeptidase U or plasminogen activator inhibitor-1 (PA-1).

26. The combination according to claim 25 wherein the synthetic heparin derivative is fondaparinux; the synthetic or biotechnological inhibitor of a coagulation factor is chosen from synthetic thrombin, FVIIa, FXa, FXIa, and FIXa inhibitors, and rNAPc2; the inhibitor of an ADP/ATP receptor is chosen from P2X1, P2Y1, and P2Y12; the protease activated receptor is PAR1 or PAR4; the inhibitor of a protease activated receptor is SCH 530348; and the carboxypeptidase U is CPU or TAFIa.

27. The combination according to claim 23 wherein the thrombolytic is chosen from one or more of tissue plasminogen activator, streptokinase, urokinase, prourokinase, anisoylated plasminogen-streptokinase activator complex (APSAC), an animal salivary gland plasminogen activator, microplasmin, or another plasmin variant.

28. The combination according to claim 27 wherein the tissue plasminogen activator is natural, recombinant or modified.
